(19) United States
(12) Patent Application Publication
(10) Pub. No.: US 2026/0306017 A1
(43) Pub. Date: Oct. 8, 2026

(54) ADAPTIVE THERAPEUTIC INSTRUCTION

(71) Applicant: BIOTRONIK SE & Co. KG, Berlin (DE)

(72) Inventors: Andreas ARNDT, Berlin (DE); Franziska WEGERICH, Berlin (DE); Christian MOSS, Falkensee (DE)

(73) Assignee: BIOTRONIK SE & Co. KG, Berlin (DE)

(21) Appl. No.: 19/479,959

(22) PCT Filed: Apr. 22, 2024
(86) PCT No.: PCT/EP2024/060927

§ 371 Date: Oct. 29, 2025

(30) Foreign Application Priority Data
May 12, 2023 (EP) 23173046.6

(51) Int. Cl. A61B 5/00; A61B 5/0205; A61B 5/0215; A61B 5/08; A61B 5/11; G16H 40/67; G16H 50/30

(52) U.S. Cl. CPC A61B 5/486; A61B 5/0205; A61B 5/0215; A61B 5/7275; G16H 40/67; G16H 50/30; A61B 5/0816; A61B 5/1118; A61B 5/4806

(57) ABSTRACT

The present invention relates to a system and a method for providing a therapeutic instruction to a patient, in particular for treatment of heart failure. The system comprises means for receiving, from an implanted pressure sensor, information indicative of a blood pressure of the patient, means for determining the therapeutic instruction for the patient based at least in part on a dynamic physiological model and the received information, means for updating the dynamic physiological model based at least in part on the received information, the determined therapeutic instruction or a combination thereof, and means for outputting the determined therapeutic instruction to a device accessible to the patient.

### CROSS-REFERENCE TO RELATED APPLICATIONS

This application is the United States National Phase under 35 U.S.C. § 371 of PCT International Patent Application No. PCT/EP2024/060927, filed on Apr. 22, 2024, which claims the benefit of European Patent Application No. 23173046.6, filed on May 12, 2023, the disclosures of which are hereby incorporated by reference herein in their entireties.

### TECHNICAL FIELD

The present invention relates to a system for providing a therapeutic instruction to a patient, in particular for treatment of heart failure, and a method thereof.

### BACKGROUND

Chronic heart failure (HF) affects more than 60 million people worldwide, and about three million in Germany. Even if patients with advanced HF are treated with medication according to the national and international Health Care Guideline on Chronic Heart Failure, they still suffer from fluid imbalances. This leads to one million hospital admissions each year in the USA and in Europe. In Germany, the costs of HF amount to €3.2 billion, of which 45% are due to inpatient hospital care. By implanting a pulmonary artery pressure sensor (PAPS), it is possible to detect and treat abnormalities before they become clinically manifest. If the sensor measures an excess of fluid volume, assigned medical teams should contact the patient and adjust the therapy.

Initial clinical experience with PAPS has been available for several years. In general, the implanted pressure sensor is read manually by the patient at home and the data is sent to a database. If a critical value of the PAP (pulmonary artery blood pressure) is exceeded, an alert is displayed on a computer that can be viewed by the attending physician and a contact with the patient is triggered. In a structured telephone interview, the patient is asked about potential triggers, and an existing therapy regime is adjusted if necessary. One key aspect for treatment of HF focuses on volume regulation through defined fluid intake and diuretics. However, the review of each patient's data and the adjustment of the therapy requires a high personnel effort. Further, the resulting physician-patient interactions are time-consuming and cannot be scaled arbitrarily regarding the number of patients and the frequency of contacts. There have been attempts in the prior art to improve medical treatments for patients affected by heart failure and to reduce the number and duration of physician-patient interactions by relying on homecare systems.

For example, US 2013/0178786 A1 discloses an apparatus comprising one or more physiological sensing circuits that generate a sensed physiological signal and at least one of the physiological sensing circuits is implantable, a measurement circuit configured to recurrently measure one or more physiological parameters that indicate a status of heart failure of the subject and a comparison circuit configured to compare the one or more physiological parameter measurements to one or more physiological parameter target values. The apparatus further comprises a therapy circuit configured to control delivery of one or more drugs to treat heart failure, and a control circuit in electrical communication with the comparison circuit and the therapy circuit and configured to recurrently adjust delivery of drug therapy according to the comparison of the measured physiological parameters to the physiological parameter targets. However, since the fixed physiological parameter target values require to be updated over time, frequent physician-patient interactions are still necessary.

Alternatively, instead of relying on fixed physiological parameter target values, U.S. Pat. No. 8,585,604 B2 discloses a creation of a therapy regimen, e.g., a contingent medication prescription, which is automatically distributed to a patient via an integrated patient care system. A clinician may create a limited set of therapy instructions based on associating patient conditions with one or more therapy regimens, e.g., medication prescriptions. The integrated patient care system may present historical condition data to the clinician to aid the clinician with creating and/or updating the therapy instructions specific to the patient. A therapy module of the integrated patient care system may use the therapy instructions to automatically select a therapy regimen from the therapy instructions set based on a patient condition detected based on a physiological parameter, e.g., pulmonary artery pressure. However, creating and updating the therapy instructions and the corresponding therapy regimen of each patient in a regular manner is very costly and limits the scalability of the described care system.

Similarly, US 2018/0168463 A1 relates to exercise triggered cardiovascular pressure measurement. Based on the data, a patient may determine whether or not to take a particular medication, such as a diuretic medication, or a blood thinner or a blood pressure medication, and for example may allow the patient to adjust a dosage of a medication that is to be taken based on the blood pressure data provided by the system. In addition, the data may also be useful and convenient for both the patient and another person, such as a clinician or a physician caring for the patient, by allowing pulmonary blood pressure measurements to be taken at a particular time and under particular conditions, such as when the patient is at a doctor's appointment and in the physical presence of the clinician or the physician. However, independent adjustments of the prescribed medication require experience and knowledge of the respective medical condition. Especially for elderly people, the risk of misjudgment leading to potentially dangerous incorrect dosages is highly relevant.

Thus, the known systems and methods still require frequent physician-patient interaction and cause considerable amount of work for the physician. Furthermore, early and improved detection of therapy adjustments are still missing.

It may therefore be considered as a problem underlying the present invention to provide an improved system for providing a therapeutic instruction to a patient that avoids at least in part the difficulties encountered in the prior art.

The above problem may at least partly be solved by a system for providing a therapeutic instruction to a patient, in particular for treatment of heart failure. It may comprise means for receiving, from an implanted pressure sensor, information indicative of a blood pressure (e.g. one or more blood pressure values) of the patient, and means for determining the therapeutic instruction for the patient based at least in part on a dynamic physiological model and the received information. The system may further comprise means for (automatically) updating the dynamic physiological model based at least in part on the received information, the determined therapeutic instruction or a combination thereof, and means for outputting the determined therapeutic instruction to a device accessible to the patient. Such a system may allow for an adaptively adjusted and optimized therapeutic instruction for the patient so that the above outlined disadvantages of the prior art may at least partly be overcome.

In this manner, the above system can provide one or more therapeutic instructions to the patient merely based on the predictions made by the dynamic physiological model based on measurements, past therapy instructions and optional supplemental information such as dietary observations, compliance with past therapy instructions and alterations of the comorbidities. Thus, time-consuming and costly interactions with an attending physician can be omitted. Based thereon, the system can provide an advanced hemodynamic monitoring for a larger group of patients per healthcare professional or physician. This also reduces the monitoring costs per patient immensely. Another advantage of the above system concerns the timing aspect of a detection of irregularities of a patient's condition. In particular, the provided system may adapt a dosage of an already prescribed medication or may even change a prescribed drug based on the received information, e.g. the information indicative of the blood pressure of the patient, even before the patient can perceive any negative or uncomfortable signs or symptoms. A fast response time can improve quality of living greatly and may further lead to less hospitalizations and less severe medical emergencies.

### SUMMARY

In some embodiments, the system may comprise or be implemented by a server comprising at least a database, a computing unit and a transceiver unit. The server may be personally owned by the patient, e.g. in form of a personal computer, included in the device accessible to the patient or similar, or it may be a remotely located server accessed over a network, e.g. via an internet or mobile network connection. In either case, the server may receive the information from the implanted pressure sensor of the patient via the means for receiving, e.g. the transceiver unit, and may store the respective data in the database. The means for determining, e.g. the computing unit, may utilize the data stored on the database for determining the therapeutic instruction. The database may comprise at least the recently received information, previously received information, the dynamic physiological model including e.g. required parameters or variables, and previously determined therapeutic instructions if available. By not only relying on the recently received information, but also including formerly received information and previously determined therapeutic instructions, the dynamic physiological model may be updated utilizing the means for updating, e.g. the computing unit. For example, one or more parameters of the model may be altered based on the updating. The means for outputting, e.g. the transceiver unit, may further output the determined therapeutic instruction to the device accessible to the patient. Additionally or alternatively, the system may further comprise an implantable pressor sensor, e.g. the implanted pressure sensor, and/or the device accessible to the patient. Additionally, the system may further comprise a means to communicate bidirectionally with the patient and to enquire supplemental information from the patient such as dietary observations, compliance with past therapy instructions and/or alterations of the comorbidities.

The blood pressure may correspond to a pulmonary artery pressure, a pressure in the left or right atrium or a filling pressure towards the left or right ventricle. In general, heart failure corresponds to a situation, in which the heart is unable to pump sufficiently to maintain blood flow to meet the body tissues' needs for metabolism. One consequence of an insufficient blood flow can be an increased cardiac filling pressure or a blood backlog which the heart is not capable to pump away. Therefore, monitoring the blood pressure of the lung circulation, in particular the pulmonary artery pressure, the pressure in a left atrium or the filling pressure towards the left ventricle greatly improves detection and/or monitoring of early symptoms of heart failure reliably. The implanted pressure sensor may correspondingly be implanted in a pulmonary artery, for example.

A key principle of hemodynamically guided pharmacological therapy of heart failure is the dosage of medication to manage the patient's fluid balance depending on a filling pressure of the left heart, which can be determined as described above. Thereby, the filling pressure corresponds to both, the measured and controlled variable, and the medication corresponds to the manipulated variable, while the controlled system is the human body. The body reacts to an increased medication dose, for example, by excretion and reduced absorption of fluid. As a result, the filling pressure may decrease.

The therapeutic instruction may comprise at least one of: a recommendation regarding liquid intake, a recommendation regarding intake of medication, in particular diuretics (and/or RAAS inhibitors, etc.), a dietary recommendation, in particular regarding salt intake, or a combination thereof. As noted above, heart failure can result in an increased pulmonary artery pressure for an extended period, typically above 25 mmHg at rest or above 30 mmHg during exercise. The therapeutic instructions can provide an efficient and reliable method for effectively reducing the severity of the medical condition and/or alleviate symptoms associated with heart failure by regulating the patient's fluid balance. Thereby, the quality of life of the patient can be improved.

In the prior art, the dosage of medications has been done manually by the attending physician depending on the provided information, e.g. by an implanted sensor. The physician applies his expertise and experience to determine the dosage change depending on the measured value or the change in the measured value. The present invention enables this elaborate task to be performed by an automatic controller based on the dynamic physiological model. For this, the knowledge of a characteristic of the controlled system, i.e. the human body, is necessary. On the one hand, the static relationship between drug dosage and filling pressure or filling pressure change is important. It is known that the dose-response relationship between diuretics and excretion volume is strongly nonlinear. This relationship is patient-specific and time-varying. On the other hand, the temporal dynamics between drug administration and the pressure change is important to achieve the fastest possible regulation of filling pressure. The dynamics is also patient-specific and/or time-varying and possibly also nonlinear. The static and dynamic dose-response relationship can be described with physiological models. Both pharmacokinetic and pharmacodynamic models (PK/PD models) can be part of the physiological model.

The dynamic physiological model according to the present invention may be based at least in part on a pathophysiological model of the patient's fluid balance comprising a pharmacokinetic model, a pharmacodynamic model, a model of the relationship of fluid and salt intake with cardiac filling pressures, a model of renal function in response to medication change, a model of the response of electrolytes on medication change, a model of the systemic arterial blood pressure on medication change or a combination thereof. The dynamic physiological model may be a physiology-based model parametric model, a non-parametric model or a data driven model. The structure and parameters of these models can be determined by system identification methods. For this purpose, clinical studies can be performed to determine data for the dose-response or action-response relationship. Based thereon, generic base-line models can be derived. Online identification procedures for the parameters of the models may be required to account for interpatient variability and/or change in dynamics over time. These procedures can provide up-to-date, patient-specific models for the dose-response relationship, in particular for diuretics or action-response relationship for the remaining variables, e.g. fluid intake on filling pressure.

In some embodiments, the dynamic physiological model may at least be adapted for determining a dose-response relationship between one of: an intake of a medication, in particular a diuretic (and/or an RAAS inhibitor), and the blood pressure, in particular the pulmonary artery blood pressure, of the patient; a liquid intake and the blood pressure, in particular the pulmonary artery blood pressure, of the patient; or a combination thereof. Thus, by relying on the dynamic physiological model according to the present invention, frequent patient-physician interactions can be omitted.

In some embodiments, the dynamic physiological model may predict the future evolution of the input variables, in particular the blood pressure as pulmonary artery blood pressure and/or atrial blood pressure, creatinine, electrolytes. Such an embodiment would allow a forecast for the therapeutic instructions.

Additionally or alternatively, determining the therapeutic instruction for the patient and/or updating the dynamic physiological model may be autonomously executed by the system, in particular without interacting with a healthcare professional or an attending physician. In this manner, an advanced hemodynamic monitoring for a larger group of patients per healthcare professional or physician may be provided. Additionally, without requiring an appointment with the attending physician, a response time for adjusting the patient's medication can be severely reduced.

The system may further comprise means for receiving, from the implanted pressure sensor, information indicative of at least one disturbance effect. An accuracy of the information indicative of the blood pressure of the patient can be enhanced based at least in part on the information indicative of the at least one disturbance effect. The disturbance effect may coexist during a determination of the information indicative of the blood pressure of the patient. Additionally or alternatively, the disturbance effect may relate to a body position of the patient, a physical activity of the patient, one or more breathing cycles of the patient, sleep-related respiratory disorders of the patient, for example one or more breathing pauses of the patient asleep, or a combination thereof. By this, errors in measurement in determining the blood pressure of the patient based on the implanted pressure sensor can be reduced. For example, prior to inputting the information indicative of a blood pressure of the patient received by the system into the dynamic physiological model, known errors in measurement and/or disturbance effects may be corrected/compensated based on the information indicative of the at least one disturbance effect. Similarly, relying on an average value of multiple measurements, in particular multiple measurements taken at night while the patient is asleep, may also be applicable for increasing the accuracy of the determined pressure value. As a result, a corrected, i.e. more accurate, information indicative of a blood pressure of the patient can be entered into the dynamic physiological model for determining the therapeutic instruction. This results in a more reliable therapeutic instruction for each individual patient.

The device accessible to the patient may comprise a smartphone, a tablet computer or a personal computer. For example, a mobile application and/or a software may be installed on the patient's smartphone, tablet computer or personal computer for displaying the determined therapeutic instruction. By this, the patient may utilize already owned devices for receiving the determined therapeutic instruction, which not only minimizes the costs for the health care system to utilize the system according to the present invention but also reduces the carbon footprint by reusing existing hardware.

The system may further comprise means for receiving, from the device accessible to the patient, an ambient pressure of the device. For most known implantable pressure sensors, the information indicative of the blood pressure may relate to an absolute pressure value. Utilizing the received ambient pressure, the system can determine the relative blood pressure of the patient. In other words, disturbance effects based on a change of ambient pressure, e.g. while traveling, can be compensated. This increases the accuracy of the information indicative of a blood pressure entered into the dynamic physiological model.

In some embodiments, the device accessible to the patient, e.g., if carried by the patient during the day, can also record a pressure profile over the entire day. By this, a direct relationship of determined absolute pressure values measured in the body with the ambient pressure values determined based on the patient's device becomes possible in a time-synchronized manner.

In some embodiments, the system may further comprise means for receiving, from the device accessible to the patient, (supplemental) information about a current condition of the patient, wherein the information can be entered into the device by the patient. In this case, the dynamic physiological model may be adapted to also take the received information about the current condition of the patient into account for determining the therapeutic instruction. The (supplemental) information may indicate at least one of: an affliction, a change in liquid intake, a change in diet, a change in intake of medication, a result of a laboratory test, a self-conducted measurement of a body weight, a self-conducted measurement of a blood pressure, in particular an artery blood pressure of the systemic circulation of the patient, a self-conducted measurement of an amount of excreted urine, or a combination thereof. In this manner, the dynamic physiological model can be enabled to take multiple factors into account for determining the therapeutic instruction. The more information about the patient can be entered into the dynamic physiological model, the more reliable and stable the determined therapeutic instruction can get. For example, if the patient alters his/her behavior, e.g. based on a diet change, an upcoming illness, celebrations etc., the system may recognize unnormal changes in pressure. If these unnormal changes in pressure would surpass a threshold value, the system could be adapted to alert the attending physician. However, if the source/reason for the changes are entered into the dynamic physiological model, and if the dynamic physiological model knows how to handle these changes safely, intervention of the physician may be omitted.

The system may further comprise means for verifying the information about a current condition of the patient based at least in part on a plausibility check, in particular in the form of checking that the observed time course of the pressure meets the expected time course following an intervention. Additionally, the system may comprise a security query. For example, the mobile application/software on the patient device for entering information provided for the dynamic physiological model may be password protected, fingerprint protected, gesture protected or protected by face recognition. Similar security queries known to the skilled person are also applicable. Additionally or alternatively, the system may request the patient to confirm the medication taken and/or may remind the patient to take the medication.

The system may further comprise means for outputting the determined therapy instruction to a device accessible to an attending physician. By this, the attending physician is enabled to supervise the therapeutic instructions determined by the system according to the present invention at any time. Furthermore, if the information entered into the dynamic physiological model cannot be processed or exceed safety thresholds, the attending physician can be alerted immediately.

In another aspect, the present invention provides a method for providing a therapeutic instruction to a patient, in particular for treatment of heart failure. The method may comprise receiving, from an implanted pressure sensor, information indicative of a blood pressure of the patient and determining the therapeutic instruction for the patient based at least in part on a dynamic physiological model and the received information. The method may further comprise updating the dynamic physiological model based at least in part on the received information, the determined therapeutic instruction or a combination thereof, and outputting the determined therapeutic instruction to a device accessible to the patient.

While the advantages, technical effects and additional aspects noted above regarding a system for providing a therapeutic instruction to a patient, also apply for the method for providing a therapeutic instruction to a patient, these advantages and technical effects are not repeated explicitly for reasons of brevity and conciseness.

The blood pressure may correspond to a pulmonary artery pressure, a pressure in a left atrium or a filling pressure towards the left ventricle.

The therapeutic instruction may comprise at least one of: a recommendation regarding liquid intake, a recommendation regarding intake of medication, in particular diuretics (and/or RAAS inhibitors, etc.), a dietary recommendation, in particular regarding salt intake, or a combination thereof.

The dynamic physiological model may be based at least in part on a pharmacokinetic model, a pharmacodynamic model, a model of the relationship of fluid and salt intake with cardiac filling pressures, a model of renal function in response to medication change, a model of the response of electrolytes on medication change, a model of the systemic arterial blood pressure on medication change or a combination thereof. It may be a physiology-based model or a data driven model. It may be a linear model or a non-linear model.

The dynamic physiological model may be at least adapted for determining a dose-response relationship between one of: an intake of a medication, in particular a diuretic, and the blood pressure of the patient, a liquid intake and the blood pressure of the patient or a combination thereof.

The dynamic physiological model (130) may further predict the future evolution of the input variables, in particular the blood pressure as pulmonary artery blood pressure and/or atrial blood pressure, creatinine, electrolytes.

The method may further comprise receiving, from the implanted pressure sensor, information indicative of at least one disturbance effect, wherein an accuracy of the information indicative of the blood pressure of the patient is enhanced based at least in part on the information indicative of the at least one disturbance effect.

The disturbance effect may coexist during a determination of the information indicative of the blood pressure of the patient and/or wherein the disturbance effect relates to a body position of the patient, an activity of the patient, one or more breathing cycles of the patient, one or more breathing pauses of the patient while sleeping or a combination thereof.

The device accessible to the patient may comprise a smartphone, a tablet computer or a personal computer.

The method may further comprise receiving, from the device accessible to the patient, an ambient pressure of the device.

The method may further comprise receiving, from the device accessible to the patient, information about a current condition of the patient, wherein the information was entered into the device by the patient.

The information may indicate at least one of: afflictions, changes in liquid intake, changes in diet, changes in intake of medication, results of laboratory tests, self-conducted measurements of a body weight, self-conducted measurements of a blood pressure, in particular an artery blood pressure of the systemic circulation of the patient, self-conducted measurements of an amount of excreted urine, or a combination thereof.

The method may further comprise verifying the information about a current condition of the patient based at least in part on a plausibility check and/or a security query.

The method may further comprise outputting the determined therapy instruction to a device accessible to an attending physician.

Determining the therapeutic instruction for the patient and/or updating the dynamic physiological model may be autonomously executed by the method, in particular without interacting with a healthcare professional or an attending physician.

It is noted that all aspects and functions described herein with reference to a system or device may be implemented as steps of a method. Moreover, it is understood that all method steps described herein may also be implemented by systems and devices having corresponding means to carry out these steps.

### BRIEF DESCRIPTION OF THE DRAWINGS

Aspects of the present invention are described in more detail in the following by reference to the accompanying Figures. These Figures show:

FIG. 1 an illustration of a system for providing an automated therapeutic instruction for a patient affected by heart failure according to the present invention;

FIG. 2 a block diagram of the dynamic physiological model; and

FIG. 3 a flow chart illustrating a method for providing a therapeutic instruction to a patient according to the present invention.

### DETAILED DESCRIPTION

In the following, exemplary embodiments of the present invention are described in more detail, with reference to a system and method for providing a therapeutic instruction to a patient. While specific feature combinations are described in the following with respect to the exemplary embodiments of the present invention, it is to be understood that the disclosure is not limited to such embodiments. In particular, not all features have to be present for realizing the invention, and the embodiments may be modified by combining certain features of one embodiment with one or more features of another embodiment.

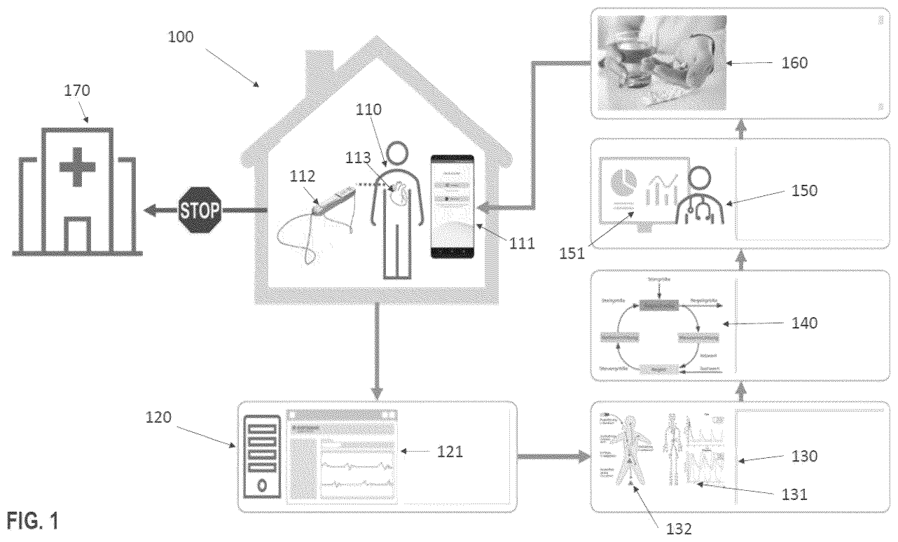

FIG. 1 depicts an embodiment of a system 100, in particular a homecare system, for a patient 110. A pressure sensor 112, which can be a component of system 100 or an individual element, has been implanted into patient 110. The pressure sensor 112 may regularly measure a blood pressure corresponding to a pulmonary artery pressure, a pressure in a left or right atrium of heart 113 or a filling pressure towards the left or right ventricle of heart 113 and can be arranged inside a pulmonary artery or left or right atrium of heart 113. For a valid automatic measurement, correction of the influence of body posture and physical activity of patient 110 on the pressure signal may be provided by measurements with an accelerometer integrated in pressure sensor 112 and correction of the influence of breathing and sleep-related respiratory disorders. Furthermore, a disturbance signal suppression can be carried out by recording several measured pressure values while patient 110 is asleep.

The measured blood pressure or respective information indicative thereof is transmitted to a patient device 111 of patient 110. Patient device 111 may for example be a smartphone, tablet computer, a personal computer or similar (not shown in FIG. 1), which are accessible to patient 110. Patient device 111 can further record an ambient pressure and utilize this ambient pressure to compensate for the determined absolute blood pressure measured in the body. If patient device 111 is carried by patient 110 regularly, patient device 111 can also record a pressure profile over the entire day so that the absolute blood pressures measured in patient's 110 body can be directly related to/corrected by the ambient pressures.

Patient device 111 is further connected to a database 121 stored on a remotely located server 120 via e.g. an internet connection, Wi-Fi, a cellular network, e.g. via LTE or 5G, or similar. Server 120 may also be seen as part of the system 100. Patient device 111 automatically transfers the pressure data and optionally further information, as for example disturbance variables, to database 121, where the data are algorithmically processed. First, raw and trend data can be filtered and preprocessed. For this purpose, disturbance variables like body posture, physical activity and respiration are compensated by the respective measured disturbance signals, which are received by server 120.

Subsequently, a dose-response relationship can be determined patient-specifically based on the compensated measured values and any past therapeutic instructions 160 if available. For this purpose, non-linear pharmacokinetics (PK) 132 and pharmacodynamics (PD) 131 models with a given structure but variable parameters can be utilized as basis for dynamic physiological model 130. Dynamic physiological model 130 can be executed on server 120. The parameters can be determined by a nonlinear parameter estimation procedure. The measured controlled variable, i.e. the compensated pressure values, can be understood as an input of a control algorithm 140 that finally determines the therapeutic instruction 160. The characteristics of the control algorithm 140 can be adapted according to the patient-specific and/or time-varying dynamic physiological model 130. The output variable of control algorithm 140 corresponds to therapeutic instruction 160, for example a specific medication. The specific medication may be an instruction for taking a diuretic, such as furosemide, for example.

Dynamic physiological model 130, the determined therapeutic instruction 160 and/or control algorithm 140 can be supervised and adapted, optionally, if required, by attending physician 150 or a healthcare professional. For this purpose, server 120 may transmit the corresponding data stored in database 121 to a device 151 accessible by physician 150, e.g. a dashboard. Alternatively, physician 150 or a respective healthcare professional can login directly on server 120 at any time. If considered necessary, physician 150 or the respective healthcare professional may be able to change the determined therapeutic instruction 160 and/or adapt dynamic physiological model 130 and/or even improve control algorithm 140. In any case, control algorithm 140 may prompt the attending physician 150 to intervene if the automatic control algorithm 140 encounters unforeseen reactions of the measured variables to the determined therapeutic instructions or detects any further emergency of patient 110.

After the control algorithm has determined therapeutic instruction 160, which may have been altered by physician 150 or may be solely determined based on dynamic physiological model 130, control algorithm 140 and the compensated pressure values, server 120 may transmit the determined therapeutic instruction 160 to patient device 111. Additionally, information about the current health condition of patient 110 may also be transmitted to and displayed on patient device 111. For example, patient device 111 may present blood pressure trend data and/or a traffic light system to patient 110. In the traffic light system green may indicate a well-adjusted fluid-balance, yellow may indicate minor issues or unexpected responses of patient 110 to recent therapeutic instructions 160, which the algorithm 140 tries to regulate/compensate itself and red may indicate more severe problems, for which an interaction with the attending physician 150 may be recommended.

In this manner, system 100 may provide patient 110 with regularly updated and patient-specific therapeutic instructions 160 in a cost-efficient and effective manner. System 100 may already detect early changes in patient's 110 medical condition even before any symptoms are perceivable by patient 110 and may adapt the medication respectively. By this, hospitalization 170 and elaborate patient-physician interactions may be omitted.

Optionally, patient 110 may be requested to enter additional information via an automated dialog using patient device 111. For example, patient 110 may enter one or more of the following, either spontaneously or on request: Complaints, changes in drinking quantity, diet and medication, answers to inquiries, laboratory examination results, accompanying measurements taken by the patient (weight, artery blood pressure, urine quantity per day). This information, as well as the pressure measurement data, can be included into dynamic physiological model 130 and automatic control algorithm 140 for determining therapeutic instruction 160. Particularly in this case, therapeutic instruction 160 can also include further recommendations like e.g. fluid intake and nutrition optimizations, e.g. regarding salt intake.

Furthermore, the information entered by patient 110 can be processed on patient device 111, on server 120 or directly in database 121 so that plausibility checks, security queries and/or the control of compliance with the instructions can be carried out. This can also include tracking patient compliance and promoting compliance through reminder functions via patient device 111.

As an alternative to the above described estimating patient-specific dynamic physiological model 130, a therapeutic instruction can also be determined in a model-free manner, using artificial intelligence methods such as reinforcement learning. This would require a training phase at the beginning of therapy. For example, the training may be based on historic patient data or it may reflect current practice by physicians (e.g. physicians may be presented with random data and may enter corresponding therapeutic instructions from which the artificial intelligence would learn). Therapeutic instruction 160 or any change of medication can similarly be transferred to patient device 111 as described above.

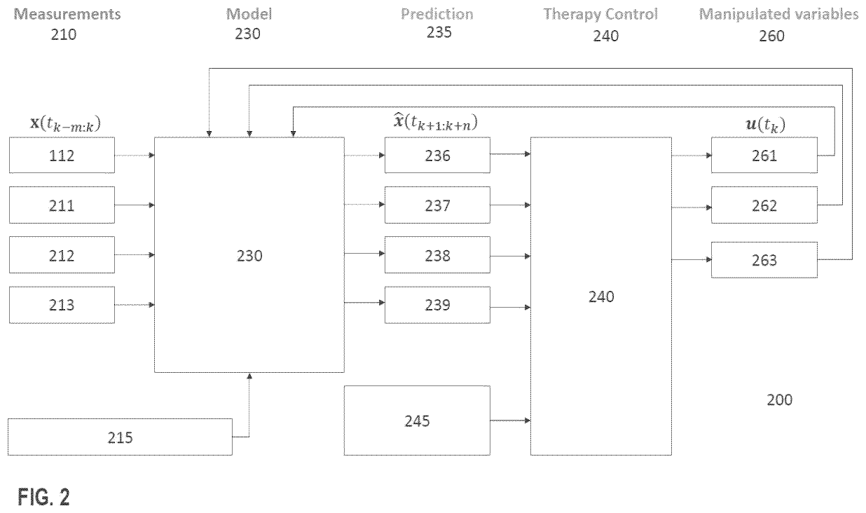

FIG. 2 depicts a block diagram of the structure of the dynamic physiologic model 130 and the automatic therapy control system 140. The dynamic physiological model 130 is represented by the model for fluid management in heart failure 230 while the automatic therapy control system 140 is represented by the therapy module 240. The model 230 receives its inputs from measurements of physiologic variables 210. One such measurement is made by pressure sensor 112. Optionally, additional measurements like arterial blood pressure (systemic blood pressure) 211, creatinine point-of-care test 212 and electrolytes 213 (determined in a laboratory test) can be inputs to model 230. The model 230 may optionally receive patient reported information 215. Additionally, the model 230 receives past therapy instructions 260. Based on this information model 230 predicts the time course of the measured variables 210 and provides the prediction results 235 to the therapy module 240. Therapy module 240 additionally receives user selected ranges 245 for the physiological variables 210 (pulmonary artery blood pressure, atrial blood pressure, creatinine, electrolytes). Therapy module 240 calculates the manipulated variables 260 as a representation of the therapy instructions 160. These can be dosage of diuretics 261, RAAS inhibitors 262 and dietary recommendations 263 etc.

By using a predictive model 230 both the automatic controller 240 will be provided with a comprehensive analysis of the future evolution of the patient's physiologic variables and optimal therapy instructions can be automatically computed. In case the physician wants to supervise the system 200 he can use the predictions 235 of the input variables as pulmonary artery blood pressure 236, atrial blood pressure 237, creatinine 238 and/or electrolytes 239 for decision making.

Model 230 incorporates physiologic relationships like the dose-response relationship, pharmacokinetics (PK) 132 and pharmacodynamics (PD) 131 models. It can be a physiology-based model, a non-parametric model or an artificial neural network etc.

The characteristics of the automatic controller 240 can be adapted according to the patient-specific and/or time-varying dynamic physiological model 230.

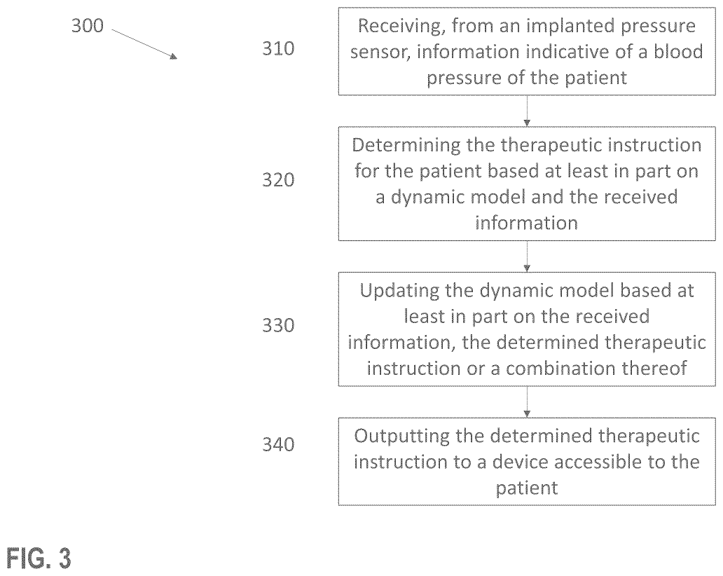

FIG. 3 depicts an embodiment of a method 300 for providing a therapeutic instruction to a patient. In step 310, method 300 comprises receiving, from an implanted pressure sensor, information indicative of a blood pressure of the patient. In step 320, method 300 comprises determining the therapeutic instruction for the patient based at least in part on a dynamic physiological model and the received information. In step 330, method 300 comprises updating the dynamic physiological model based at least in part on the received information, the determined therapeutic instruction or a combination thereof. In step 340, method 300 comprises outputting the determined therapeutic instruction to a device accessible to the patient.

Further examples, facilitating the understanding of the invention are provided below:
1. A system (100) for providing a therapeutic instruction (160) to a patient (110), in particular for treatment of heart failure, comprising means for:
   a) receiving, from an implanted pressure sensor (112), information indicative of a physiologic signal, especially a blood pressure of the patient (110);
   b) determining the therapeutic instruction (160) for the patient (110) based at least in part on a dynamic physiological model (130), the received information and the predictions thereof;
   c) updating the dynamic physiological model (130) based at least in part on the received information, the determined therapeutic instruction (160) or a combination thereof; and
   e) outputting the determined therapeutic instruction (160) to a device (111) accessible to the patient (110).
2. The system (100) according to example 1, wherein the blood pressure corresponds to a pulmonary artery pressure, a pressure in a left or right atrium or a filling pressure towards the left or right ventricle.
3. The system (100) according to example 1 or 2, wherein the therapeutic instruction (160) comprises at least one of: a recommendation regarding liquid intake, a recommendation regarding intake of medication, in particular diuretics, a dietary recommendation, in particular regarding salt intake, or a combination thereof.
4. The system (100) according to one of examples 1 to 3, wherein the dynamic physiological model (130) is based at least in part on a pharmacokinetic model (132), a pharmacodynamic physiological model (131), a model of the relationship of fluid and salt intake with cardiac filling pressures, a model of renal function in response to medication change, a model of the response of electrolytes on medication change, a model of the systemic arterial blood pressure on medication change or a combination thereof.
5. The system (100) according to one of examples 1 to 4, wherein the dynamic physiological model (130) is at least adapted for determining a dose-response relationship between one of: an intake of a medication, in particular a diuretic, and the blood pressure of the patient (110), a liquid intake and the blood pressure of the patient (110) or a combination thereof.
6. The system (100) according to one of examples 1 to 5, wherein the dynamic physiological model (130) predicts the future evolution of the input variables, in particular the blood pressure as pulmonary artery blood pressure and/or atrial blood pressure, creatinine, electrolytes
7. The system (100) according to one of examples 1 to 6, further comprising means for receiving, from the implanted pressure sensor (112), information indicative of at least one disturbance effect, wherein an accuracy of the information indicative of the blood pressure of the patient (110) is enhanced based at least in part on the information indicative of the at least one disturbance effect.
8. The system (100) according to the previous example, wherein the disturbance effect coexists during a determination of the information indicative of the blood pressure of the patient (110) and/or wherein the disturbance effect relates to a body position of the patient (110), an activity of the patient (110), one or more breathing cycles of the patient (110), one or more breathing pauses of the patient (110) while sleeping or a combination thereof.
9. The system (100) according to one of examples 1 to 8, wherein the device (111) accessible to the patient (110) comprises a smartphone, a tablet computer, a personal computer or a dedicated device.
10. The system (100) according to one of examples 1 to 9, further comprising means for receiving, from the device (111) accessible to the patient (110), an ambient pressure of the device (111).
11. The system (100) according to one of the examples 1 to 10, further comprising means for receiving, from the device (111) accessible to the patient (110), information about a current condition of the patient (110), wherein the information was entered into the device (111) by the patient (110) either spontaneously or upon request of the health care provider.
12. The system (100) according to the previous example, wherein the information indicates at least one of: afflictions, changes in liquid intake, changes in diet, changes in intake of medication, results of laboratory tests, self-conducted measurements of a body weight, self-conducted measurements of a blood pressure, in particular an artery blood pressure of a systemic circulation of the patient (110), self-conducted measurements of an amount of excreted urine, or a combination thereof.
13. The system (100) according to example 10 or 12, further comprising means for verifying the information about a current condition of the patient (110) based at least in part on a plausibility check and/or a security query.
14. The system (100) according to one of examples 1 to 13, further comprising means for outputting the determined therapy instruction to a device (151) accessible to an attending physician (150).
15. The system (100) according to one of examples 1 to 14, wherein determining the therapeutic instruction (160) for the patient (110) and/or updating the dynamic physiological model (130) is autonomously executed by the system (100), in particular without interacting with a healthcare professional or an attending physician (150).
16. A method (300) for providing a therapeutic instruction (160) to a patient (110), in particular for treatment of heart failure (300), comprising:
   a) receiving (310), from an implanted pressure sensor (112), information indicative of a physiologic signal, especially a blood pressure of the patient (110);
   b) determining (320) the therapeutic instruction (160) for the patient (110) based at least in part on a dynamic physiological model (130) and the received information and the predictions thereof;
   c) updating (330) the dynamic physiological model (130) based at least in part on the received information, the determined therapeutic instruction (160) or a combination thereof; and
   c) outputting (340) the determined therapeutic instruction (160) to a device (111) accessible to the patient (110).
17. The method (300) according to example 16, wherein the blood pressure corresponds to a pulmonary artery pressure, a pressure in a left atrium or a filling pressure towards the left ventricle.
18. The method (300) according to example 16 or 17, wherein the therapeutic instruction (160) comprises at least one of: a recommendation regarding liquid intake, a recommendation regarding intake of medication, in particular diuretics, a dietary recommendation, in particular regarding salt intake, or a combination thereof.
19. The method (300) according to example 16 to 18, wherein the dynamic physiological model (130) predicts the future evolution of the input variables, in particular the blood pressure as pulmonary artery blood pressure and/or atrial blood pressure, creatinine, electrolytes.
20. The method (300) according to one of examples 16 to 19, wherein the dynamic physiological model (130) is based at least in part on a pharmacokinetic model (132), a pharmacodynamic physiological model (131), a non-linear model or a combination thereof.
21. The method (300) according to one of examples 16 to 20, wherein the dynamic physiological model (130) is at least adapted for determining a dose-response relationship between one of: an intake of a medication, in particular a diuretic, and the blood pressure of the patient (110), a liquid intake and the blood pressure of the patient (110) or a combination thereof.
22. The method (300) according to one of examples 16 to 21, further comprising the step of receiving, from the implanted pressure sensor (112), information indicative of at least one disturbance effect, wherein an accuracy of the information indicative of the blood pressure of the patient (110) is enhanced based at least in part on the information indicative of the at least one disturbance effect.
23. The method (300) according to the previous example, wherein the disturbance effect coexists during a determination of the information indicative of the blood pressure of the patient (110) and/or wherein the disturbance effect relates to a body position of the patient (110), an activity of the patient (110), one or more breathing cycles of the patient (110), one or more breathing pauses of the patient (110) while sleeping or a combination thereof.
24. The method (300) according to one of examples 16 to 23, wherein the device (111) accessible to the patient (110) comprises a smartphone, a tablet computer or a personal computer.
25. The method (300) according to one of examples 16 to 24, further comprising receiving, from the device (111) accessible to the patient (110), an ambient pressure of the device (111).
26. The method (300) according to one of the examples 16 to 25, further comprising receiving, from the device (111) accessible to the patient (110), information about a current condition of the patient (110), wherein the information was entered into the device (111) by the patient (110).
27. The method (300) according to the previous example, wherein the information indicates at least one of: an affliction, a change in liquid intake, a change in diet, a change in intake of medication, a result of a laboratory test, a self-conducted measurement of a body weight, a self-conducted measurement of a blood pressure, in particular an artery blood pressure of a systemic circulation of the patient (110), a self-conducted measurement of an amount of excreted urine, or a combination thereof.
28. The method (300) according to example 26 or 27, further comprising verifying the information about a current condition of the patient (110) based at least in part on a plausibility check and/or a security query.
29. The method (300) according to one of examples 16 to 28, further comprising outputting the determined therapy instruction to a device (151) accessible to an attending physician (150).
30. The method (300) according to one of examples 16 to 29, wherein determining the therapeutic instruction (160) for the patient (110) and/or updating the dynamic physiological model (130) is autonomously executed by a system (100) according to one of examples 1-15, in particular without interacting with a healthcare professional or an attending physician (150).

1. A system for providing a therapeutic instruction to a patient, in particular for treatment of heart failure, comprising means for:
a) receiving, from an implanted pressure sensor, information indicative of a blood pressure of the patient;
b) determining the therapeutic instruction for the patient based at least in part on a dynamic physiological model and the received information;
c) updating the dynamic physiological model based at least in part on the received information, the determined therapeutic instruction or a combination thereof; and
d) outputting the determined therapeutic instruction to a device accessible to the patient.

2. The system according to claim 1, wherein the blood pressure corresponds to a pulmonary artery pressure, a pressure in a left atrium or a filling pressure towards the left ventricle.

3. The system according to claim 1, wherein the therapeutic instruction comprises at least one of: a recommendation regarding liquid intake, a recommendation regarding intake of medication, in particular diuretics, a dietary recommendation, in particular regarding salt intake, or a combination thereof.

4. The system according to claim 1, wherein the dynamic physiological model is based at least in part on a pharmacokinetic model, a pharmacodynamic physiological model, a model of the relationship of fluid and salt intake with cardiac filling pressures, a model of renal function in response to medication change, a model of the response of electrolytes on medication change, a model of the systemic arterial blood pressure on medication change or a combination thereof.

5. The system according to claim 1, wherein the dynamic physiological model is at least adapted for determining a dose-response relationship between one of: an intake of a medication, in particular a diuretic, and the blood pressure of the patient, a liquid intake and the blood pressure of the patient or a combination thereof.

6. The system according to claim 1, wherein the dynamic physiological model predicts the future evolution of the input variables, in particular the blood pressure as pulmonary artery blood pressure and/or atrial blood pressure, creatinine, electrolytes.

7. The system according to claim 1, further comprising means for receiving, from the implanted pressure sensor, information indicative of at least one disturbance effect, wherein an accuracy of the information indicative of the blood pressure of the patient is enhanced based at least in part on the information indicative of the at least one disturbance effect.

8. The system according to claim 7, wherein the disturbance effect coexists during a determination of the information indicative of the blood pressure of the patient and/or wherein the disturbance effect relates to a body position of the patient, an activity of the patient, one or more breathing cycles of the patient, one or more breathing pauses of the patient while sleeping or a combination thereof.

9. The system according to claim 1, wherein the device accessible to the patient comprises a smartphone, a tablet computer or a personal computer.

10. The system according to claim 1, further comprising means for receiving, from the device accessible to the patient, an ambient pressure of the device.

11. The system according to claim 1, further comprising means for receiving, from the device accessible to the patient, information about a current condition of the patient, wherein the information was entered into the device by the patient.

12. The system according to claim 11, wherein the information indicates at least one of: afflictions, changes in liquid intake, changes in diet, changes in intake of medication, results of laboratory tests, self-conducted measurements of a body weight, self-conducted measurements of a blood pressure, in particular an artery blood pressure of a systemic circulation of the patient, self-conducted measurements of an amount of excreted urine, or a combination thereof.

13. The system according to claim 11, further comprising means for verifying the information about a current condition of the patient based at least in part on a plausibility check and/or a security query.

14. The system according to claim 1, further comprising means for outputting the determined therapy instruction to a device accessible to an attending physician.

15. The system according to claim 1, wherein determining the therapeutic instruction for the patient and/or updating the dynamic physiological model is autonomously executed by the system, in particular without interacting with a healthcare professional or an attending physician.
